(19) United States
(12) Patent Application Publication
(10) Pub. No.: US 2014/0066901 A1
(43) Pub. Date: Mar. 6, 2014

(54) Systems, Devices and Methods for Providing Therapy with Image Guidance

(71) Applicant: ENTrigue Surgical, Inc., San Antonio, TX (US)

(72) Inventors: Fred B. Dinger, III, San Antonio, TX (US); Prasad Nalluri, Boerne, TX (US)

(21) Appl. No.: 14/014,006

(22) Filed: Aug. 29, 2013

Related U.S. Application Data
(60) Provisional application No. 61/694,478, filed on Aug. 29, 2012.
(60) Provisional application No. 61/818,744, filed on May 2, 2013.

(51) Int. Cl. A61M 25/01

(52) U.S. Cl. CPC A61M 25/0105
USPC 604/514; 604/529; 604/103.1

(57) ABSTRACT

Systems, devices, and methods comprising an instrument for treating an anatomical opening such as a paranasal sinus.

### CROSS-REFERENCE TO RELATED APPLICATIONS

This application claims priority to U.S. Provisional Application No. 61/694,478 filed Aug. 29, 2012, and U.S. Provisional Application No. 61/818,744 filed May 2, 2013. The entire content of each of the above—referenced disclosures is specifically incorporated herein by reference without disclaimer.

### BACKGROUND OF THE INVENTION

Surgical treatments for ear, nose and throat (ENT) disorders (e.g. sinusitis) have evolved slowly. In current clinical practice, functional endoscopic sinus surgery (FESS) is used to treat disorders where mucous drainage is impaired and/or chronic infections are present. In FESS, an endoscope is inserted into the nose and, under visualization through the endoscope, the surgeon may remove diseased or hypertrophic soft tissue or bone and may enlarge the ostia of the sinuses to restore normal drainage of the sinuses. FESS procedures can be effective in the treatment of sinusitis and for the removal of tumors, polyps and other aberrant growths from the nose. Other endoscopic intranasal procedures have been used to remove pituitary tumors, to treat Graves disease (i.e., a complication of hyperthyroidism which results in protrusion of the eyes) and to bring about surgical repair of rare conditions, such as cerebrospinal fluid rhinorrhea where cerebrospinal fluid leaks into the nose.

In certain instances, sinus and ENT surgery has been performed with the assistance of electronic navigation devices (i.e., “image-guided FESS”). In typical image guided surgical procedures, integrated anatomical information is supplied through CT-scan images or other anatomical mapping data taken before the operation. Data from a preoperative CT scan or other anatomical mapping procedure is downloaded into a computer and special sensors known as localizers or location sensors are attached to the surgical instruments. Thus, using the computer, the surgeon can ascertain, in three dimensions, the precise position of each location sensor-equipped surgical instrument at any given point in time. This information, coupled with the visual observations made through the standard endoscope, can help the surgeon to carefully position the surgical instruments to avoid creating CSF leaks and to avoid causing damage to nerves or other critical structures.

Although FESS is an accepted therapy for severe sinuses, it has several shortfalls.

Often patients complain of the post-operative pain and bleeding associated with the procedure. A significant subset of patients remain symptomatic even after multiple surgeries. Since FESS is considered an option only for the most severe cases (those showing abnormalities under CT scan), a large population of patients exist that either cannot tolerate the prescribed medications or are not considered candidates for surgery. Further, because the methodologies to assess sinus disease are primarily static measurements (e.g., CT, MRI), patients whose symptoms are episodic are often simply offered drug therapy when in fact underlying mechanical factors may play a significant role in their condition. To date, there is no mechanical therapy offered for these patients, and even though they may fail pharmaceutical therapies, no other course of action is indicated. This leaves a large population of patients in need of relief, unwilling or afraid to take steroids, but not sick enough to qualify for surgery.

The need for more minimally invasive treatments of diseased paranasal sinuses has resulted in the proposal of balloon dilation methods and devices. For example, U.S. Pat. No. 2,525,183 (Robison) discloses an inflatable pressure device which can be inserted within the sinus and inflated to restore the sinus passage to normal conditions. Lanza and others have used a Fogarty balloon to dilate nasal sinus passages to enlarge the openings and restore normal mucous drainage, as described by Orlandi et al. (2001) and referenced by Lanza (2006).

U.S. Patent Publication No. 2004/0064150 A1 (Becker) and related applications disclose balloon catheters formed of a stiff hypotube to be pushed into a sinus. The balloon catheters have a stiff hypotube with a fixed pre-set angle that enables them to be pushed into the sinus. In at least some procedures wherein it is desired to position the balloon catheter in the ostium of a paranasal sinus, it is necessary to advance the balloon catheter through complicated or tortuous anatomy in order to properly position the balloon catheter within the desired sinus ostium. Also, there is a degree of individual variation in the intranasal and paranasal anatomy of human beings, thus making it difficult to design a stiff-shaft balloon catheter that is optimally shaped for use in all individuals. Indeed, rigid catheters formed of hypotubes that have pre-set angles cannot be easily adjusted by the physician to different shapes to account for individual variations in the anatomy. In view of this, the Becker patent application describes the necessity of having available a set of balloon catheters, each having a particular fixed angle so that the physician can select the appropriate catheter for the patient's anatomy. The requirement to test multiple disposable catheters for fit is likely to be very expensive and impractical. Moreover, if such catheter are disposable items (e.g., not sterilizable and reusable) the need to test and discard a number of catheters before finding one that has the ideal bend angle could be rather expensive. Furthermore, the rigidity of the catheters described by Becker may make access to certain acutely angled ostia difficult in the confined space of the nasal cavity. A further disadvantage of Becker is the inability to verify that the balloon position is in the correct location. In some anatomy where direct visualization is difficult to impossible, for example in the frontal recess, there is a risk of entering and dilating the wrong opening, which at best does not resolve the clinical symptoms and in some cases may lead to severe clinical complications.

Further, balloon dilation of the paranasal sinuses has been proposed using traditionally vascular devices and techniques. For example, European physicians have reported the use of a hydrophilic guidewire and standard PTCA balloon catheter to treat restenosis of surgically created openings in diseased frontal sinuses and stenotic nasal conae. Göttmann, D., Strohm, M., Strecker, E. P., Karlsruhe, D. E., Balloon dilatation of Recurrent Ostial Oclusion of the Frontal Sinus, Abstract No. B-0453, European Congress of Radiology (2001); Strohm, M., Göttmann, D., Treatment of Stenoses of Upper Air Routes by Balloon Dilation, Proceeding of the 83^{rd} Annual Convention of the Association of West German ENT Physicians (1999).

A system of devices utilizing this approach is described in U.S. Pat. Nos. 7,462,175 and 7,500,971. This system includes a guidewire, and a guide catheter to position a balloon catheter into the target paranasal sinus. The balloon is then inflated to dilate the nasal opening. This system provides some advantages over the rigid system described by Becker. The guide wire allows access to sinuses around tortuous anatomy, with the guide catheter providing support for the floppy guide wire and balloon catheter. This system also includes two possible methods of position verification: fluoroscopy, or a guidewire with illumination.

Clinical experience with this system has demonstrated successful access and balloon dilation of sinus passages. However, several disadvantages remain with this approach. The addition of devices such as guide wires and guide catheters to navigate and position the balloon adds significant complexity and cost to the surgical case. As described, this added cost and complexity often prohibits these prior systems to be used in conjunction with standard sinus surgery equipment and techniques, but instead be used as a stand-alone procedure for isolated disease. This factor limits the clinical utility of this prior system, for example it does not allow the concurrent removal of the uncinate process or removal of the ethmoid air cells. In addition, the techniques employed to use these prior systems are not standard to the average ENT surgeon and require extensive training. Use of the fluoroscopy system alone requires extensive and expensive additions to operating room equipment, user training, and in some cases user certification. In addition, as with the Becker system, the guide catheters are shaped with a set angle, so that access to multiple sinuses in one patient may involve the use of several devices, increasing the cost of the procedure still further. Another disadvantage with the method used to place the balloon catheter, requiring the manipulation of a guide catheter and guide wire, is that this method requires at least two hands, and sometimes a third via an assistant, thus the concurrent use of an endoscope for direct visualization, as is standard for current sinus surgical procedures, would require an assistant: further cost and personnel in the operating room.

The structure of these devices also presents disadvantages. Because of the lack of rigidity of the guidewire and guide catheter, it is impossible to precisely locate the tips of these devices in 3-D space. While this is not an issue for vascular procedures where the working space is essentially linear, this is not true for the sinus cavities. Further, the lack of rigidity of the devices also lessens the ability to push the balloon across the tight spaces often encountered in chronic sinusitis patients, which may be obstructed by scar or granulation tissue. Finally, the lack of rigidity precludes the use of most image guidance navigation systems for positioning and verifying the location of the balloon.

Maintenance of patency of the maxillary, frontal and sphenoid sinus cannot be assured by purely balloon dilating the opening, and may require stenting the dilated sinus with an expandable stent to assure patency. The stent should preferably be absorbable to eliminate the risk and cost of removing the stent after healing has occurred.

Prior systems, based on cardiovascular technology may use guide catheters and guide wires for delivery and positioning. In addition, these systems can require fluoroscopy and/or illumination devices for navigation and placement verification.

Prior devices, systems and methods have not been optimized for minimally invasive treatment of sinusitis, mucocysts, tumors, infections, hearing disorders, fractures, choanal atresia or other conditions of the paranasal sinuses, Eustachian tubes, Lachrymal ducts and other ear, nose, throat or mouth structures in which the atraumatic dilation and maintenance of these structures is desirable. Non-articulating instruments are not capable of navigating the tortuous pathway to some of these structures. Guidewire and guide catheter access to these structures may not be possible without risk of trauma to the anatomy, or in some cases may not be possible at all. Systems are needed which can provide balloon dilation devices utilizing hand-held, articulating insertion devices that enable accurate and rapid access to these anatomic structures, and allow balloon dilation as an adjunct to surgical procedures on these structures. For example, balloon dilation of sinus ostia will allow removal of diseased tissue such as tumors or cysts without additional surgical modification. Balloons can also be used to treat orbital floor fractures by providing stability to the orbital floor via the maxillary ostia without the need for rigid fixation. In addition to dilation of the sinus ostia, balloons can be used to dilate other stenotic regions such as the nasal choana to relieve nasal obstruction due to stenosis, in the Eustachian tube to relieve Eustachian tube obstruction and in the lacrimal duct to relieve epiphora.

There exists a need for a balloon dilation system which can be delivered and positioned using surgical instrumentation and techniques currently employed by ENT surgeons, and which may be articulated by the user to aid in access and positioning in confined spaces, and to account for the variety of anatomy encountered during treatment of a single patient, as well as the variety of anatomy from patient to patient. There furthermore exists a need for a balloon delivery system which does not require the use of guide catheters and/or guide wires, with associated procedure time and cost, as well as pre-requisite training and equipment. In addition, there exists a need for a balloon dilation system which can be used in conjunction with image-guidance navigation systems, and which do not require the use of position verification methods and equipment not standard to the average ENT surgeon such as fluoroscopy or illumination. Additionally, there exists a need for a system which can deliver a stent to a dilated sinus. Some or all of these needs are met with the embodiments described herein.

### SUMMARY

In general, embodiments of the present invention provide methods, devices and systems for diagnosing and/or treating conditions relating to anatomical structures. Specific embodiments provide methods, devices and systems for dilating an anatomical structure such as a body lumen. The present disclosure focuses on embodiments suitable for ear, nose and throat (ENT) applications. A skilled surgeon, however, will recognize that embodiments within the scope of the present disclosure may be used for other anatomical structures or body lumens.

Specific embodiments relate to diagnosing and/or treating conditions affecting ENT passageways. Non-limiting examples of such disorders or conditions include sinusitis, mucocysts, tumors, infections, hearing disorders, fractures, choanal atresia or other conditions of the paranasal sinuses, Eustachian tubes, lachrymal ducts, ducts of salivary glands and other ear, nose, throat or mouth structures.

In accordance with embodiments of the present invention, there are provided devices and methods wherein one or more therapeutic components as described herein are inserted into the nose, nasopharynx, paranasal sinus, Eustachian tubes, middle ear, lachrymal ducts, ducts of salivary glands or other anatomical passageways or sinuses of the ear, nose, throat or mouth to perform an interventional or surgical procedure. In specific embodiments, the therapeutic component comprises a dilator such as an inflatable balloon. In a further embodiment, the therapeutic component may also comprise a channel or passageway for the delivery of components and therapeutic agents to the anatomic passageways or sinuses.

In an exemplary embodiment, the therapeutic component will interface with a rigid or articulating insertion device. Once interfaced, the device can be easily guided into a desired location using standard surgical techniques, and without the need of other means to guide the device such as guidewires or rigid guide tubes. The handle of the insertion device can include an actuator for controlling the articulation, which will enable the therapeutic component to be positioned and articulated with one hand, leaving the second hand free for holding an endoscope as is standard for FESS surgery. The instrument can also have means for locking the articulation mechanism into certain positions, such that the instrument is effectively rigid at predetermined angles, giving it the feel of standard ENT surgical instrument and providing the ability to accurately position the tip of the device in three-dimensional space. The insertion device can also have provisions and features to enable the intra-operative tracking of the instrument tip using currently available navigation systems. Once the device is in place, the desired therapeutic effect (e.g., dilation, stent placement, etc.) can occur.

In an embodiment, the therapeutic component is disposable, and the insertion device is reusable. In another embodiment, both the therapeutic component and insertion device are disposable. In yet another embodiment, the therapeutic component and insertion device are integrally attached. In addition, the therapeutic component may include a flexible, elongate sleeve which protects the linkages when used with an articulating instrument, as well as shield the articulating links from tissue and blood penetration.

In certain embodiments, the therapeutic component and insertion device include coupling means which allows the therapeutic component to be removably attached to the insertion device, thereby making the therapeutic component interchangeable between different insertion devices during a single procedure. For example, the user may use a single therapeutic component coupled with a variety of articulating and/or rigid instruments to treat all of the sinuses for a single patient. This feature reduces the number of different devices needed for a single procedure, bringing down the cost of the procedure. In an embodiment, the coupling means is attached to an actuator for locking and unlocking the therapeutic component on to the shaft.

Additional embodiments include features on the insertion device which provide the ability to flush and or suction the ostia, or delivery therapeutic agents, using the same insertion device that delivers the therapeutic component. In addition, embodiments and methods are provided which allow use of a flexible scope to aid in placement of the therapeutic component.

Additional devices and methods provide for innovative stenting of the ostia of the paranasal sinuses. In certain embodiments, the therapeutic component comprises a stent mounted onto an inflatable balloon. The stent can be positioned with the insertion device and deployed via inflation of the balloon. In specific embodiments, the stent may comprise an expandable, biodegradable or non-biodegradable stent. In particular embodiments, the stent could have the ability to be formed to the shape of the opening such as an hour glass for the sphenoid and maxillary sinus, or an inverted tapered cylinder for the frontal sinus. The shaping may occur for example via inflation of a shaped balloon, or via other shaping methods. The stent may alternately be self-expandable and not require a balloon to be deployed. In this embodiment, the stent is positioned in a restrained configuration, for example covered by a restraining sleeve, and then deployed once properly positioned via removal of the restraining sleeve. In certain embodiments, the stent could be removed after the desired time for healing or could biodegrade once healing has taken place. Exemplary embodiments may deploy stents disclosed in U.S. Patent Publication No. 2006/0136041 (published Jun. 22, 2006), entitled “Slide-and-Lock Stent” and U.S. Patent Publication No. 2011/0152875 (published Jun. 23, 2011), entitled “Sinus Tube”, both of which are incorporated by reference herein.

A particular embodiment comprises an insertion device configured for inserting a therapeutic component into an anatomical structure, including for example, a paranasal sinus outflow tract. In specific embodiments, the sinus outflow tract may comprise the frontal recess, maxillary and sphenoid ostia and/or the infundibulum. The infundibulum is the space between the maxillary sinus ostium and the uncinate process that contributes to the outflow tract of maxillary, anterior ethmoid and frontal sinuses. In certain embodiments, therapy may be provided for a condition, e.g. sinusitis, by expanding or dilating the infundibulum with a therapeutic component. In certain embodiments, the outflow tract may be an artificial tract.

Specific embodiments comprise an insertion device configured or adapted to deliver a therapeutic component to a sinus outflow tract. In certain embodiments, the insertion device comprises: a shaft comprising a first end and a second end; an articulating portion proximal to the first end; a handle portion proximal to the second end; and a positioning member configured to move the articulating portion from a first position to a second position. In certain embodiments, the articulating portion comprises a plurality of articulating segments. In other embodiments, the articulating portion may comprise a cut tube (e.g. a spiral cut) or a coiled wire (e.g., a spring).

In particular embodiments, the articulating portion can be held in the second position when the first end of the shaft is inserted into a paranasal sinus comprising scar or granulation tissue. In specific embodiments, the articulating portion is held in the second position when the first end of the shaft is subjected to an external radial force and/or axial force of approximately 1.0, 0.9, 0.8, 0.7, 0.6, 0.5, 0.4, 0.3, 0.2, or 0.1 pounds or less. In particular embodiments, the insertion device comprises a tip that is rigid or semi-rigid that allows for insertion through scar or granulation tissue.

In certain embodiments, the shaft is approximately 1.0 mm to 5.0 mm in diameter and the tip is approximately 0.5 mm to 3.0 mm in diameter. In particular embodiments, the shaft is 1.0, 1.5, 2.0, 2.5, 3.0, 3.5, 4.0, 4.5 or 5.0 mm in diameter and the tip is 0.5, 1.0, 1.5, 2.0, 2.5, or 3.0 mm in diameter. In specific embodiments, the shaft is approximately 3.2 mm (0.125 inches) in diameter and the tip is 2.0 mm (0.080 inches) in diameter.

In particular embodiments, the articulating segments may be configured to articulate with a radius of curvature of approximately 5.0 mm to 25.0 mm. In particular embodiments, the articulating segments may be configured to articulate with a radius of curvature of approximately 5.0, 6.0, 7.0, 8.0, 9.0, 10.0, 11.0, 12.0, 13.0, 14.0, 15.0, 16.0, 17.0, 18.0, 19.0, 20.0, 21.0, 22.0, 23.0, 24.0 or 25.0 mm. In specific embodiments, the articulating segments may be configured to articulate with a radius of curvature of approximately 9.5 mm.

In specific embodiments, the shaft may be approximately 100 mm to 300 mm in length. In particular embodiments, the shaft may be approximately 100, 110, 120, 130, 140, 150, 160, 170, 180, 190, 200, 210, 220, 230, 240, 250, 260, 270, 280, 290 or 300 mm long.

In certain embodiments, the shaft may articulate so that the distal tip is oriented at an angle of approximately 40-160 degrees, more preferably 60-110 degrees from the proximal end of the shaft. In particular embodiments, the shaft may articulate so that the distal tip is oriented at an angle of approximately 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, or 110 degrees from the proximal end of the shaft. In particular embodiments, the distal tip of the shaft may be pre-set at an angle of approximately 0-30 degrees prior to further articulation of up to 110 degrees.

In exemplary embodiments, the articulating segments may be configured similar to systems disclosed in U.S. Pat. Nos. 7,553,275 and 7,670,284, each titled “Medical Device with Articulating Shaft,” which are incorporated by reference herein.

In certain embodiments, the articulating segments can include a plurality of independent pivot members and pins in an alternating configuration. In particular embodiments, each pivot member can define an opening while each pin can define a pin aperture. In specific embodiments, a first slat assembly and second slat assembly extend through the articulating segments. In certain embodiments, each of the first slat assembly and the second slat assembly is configured to push when the other of the first slat assembly and the second slat assembly pulls so as to cause the articulating segments to articulate.

In particular embodiments, the openings collectively define an outer passageway while the pin apertures collectively define an inner passageway or channel. In certain embodiments, the first slat assembly can extend through the outer passageway alongside a first side of the pins while the second slat assembly can extend through the outer passageway alongside a second side of the pins opposite the first side of the pins. In exemplary embodiments, the inner passageway or channel can provide a path for a flexible stylet, an actuator, a flexible tube, electrical wiring and/or light transmitting media, such as optical fibers, to extend through the articulating segments. The actuator may be formed with a variety of cross-sectional shapes, such as a rectangle, square, circle, etc.

In particular embodiments, the locking member comprises a pin extending from the positioning member. Certain embodiments may further comprise a location sensor configured to register the location of the first end of the shaft. Specific embodiments may comprise a therapeutic component coupled to the shaft proximal to the first end. The therapeutic component may be in fluid communication with a first coupling member configured to receive a pressurizing member, which can be a syringe in certain embodiments. The therapeutic component may be in fluid communication with a second coupling member configured to receive the shaft, and the second coupling member may comprise a pair of latching members configured to engage a flange on the shaft. The second coupling member may also comprise a pair of leverage members configured to open the latching members. Certain embodiments may comprise a sleeve extending between the therapeutic component and the coupling member, where the sleeve extends over the plurality of articulating portion.

In specific embodiments, the sleeve comprises a conduit in fluid communication with coupling member and the therapeutic component, which may be an inflatable balloon. In certain embodiments, the therapeutic component is configured to deliver fluid to the anatomical structure. In particular embodiments, a portion of the articulating portion extends into the therapeutic component.

Specific embodiments may comprise a locking member configured to lock the positioning member so that the articulating portion is held in the second position. In specific embodiments, the insertion device comprises a plurality of apertures configured for engagement with the locking member. Certain embodiments may further comprise a biasing member configured to bias the positioning member such that the locking member is engaged with one of the apertures.

Certain embodiments may include a method of providing therapy to a paranasal sinus outflow tract, where the method comprises: inserting a therapeutic component into the paranasal sinus outflow tract, where the therapeutic component is inserted into the paranasal sinus outflow tract without the use of a guide wire, cannula or guide sheath; and expanding the therapeutic component to enlarge the paranasal sinus outflow tract.

In specific embodiments, inserting the therapeutic component into the paranasal sinus outflow tract comprises providing a shaft with a distal end and an articulating portion; coupling the therapeutic component to the shaft; and inserting the distal end of the shaft into the paranasal sinus outflow tract. Particular embodiments may also comprise moving the articulating portion of the shaft from a first position to a second position; and engaging the distal end of the shaft with tissue proximal to the paranasal sinus outflow tract, where the articulating portion of the shaft remains in the second position when the distal end of the shaft engages the tissue proximal to the paranasal sinus outflow tract.

In specific embodiments, the tissue comprises scar or granulation tissue. Particular embodiments may further comprise dilating a therapeutic component proximal to the distal end of the shaft after the distal end has been inserted into a paranasal sinus. Specific embodiments may comprise tracking the location of the distal end of the shaft with a location sensor. In particular embodiments, the sinus is a frontal sinus. Certain embodiments may comprise delivering a therapeutic fluid to the paranasal sinus outflow tract.

Particular embodiments may comprise a method of dilating a paranasal sinus outflow tract, where the method comprises: inserting a therapeutic component into the paranasal sinus outflow tract, wherein the therapeutic component is coupled to a shaft with an articulating portion; expanding the therapeutic component from a first diameter to a second diameter, thereby dilating the paranasal sinus outflow tract; reducing the therapeutic component to the first diameter; and withdrawing the therapeutic component from the paranasal sinus outflow tract. In certain embodiments, the paranasal sinus outflow tract comprises granulation or scar tissue.

In certain embodiments, the shaft comprises a proximal end, a distal end, and the therapeutic component is located between the articulating portion and the distal end. In specific embodiments, inserting the therapeutic component into the paranasal sinus outflow tract comprises manipulating a positioning member configured to move the articulating portion of the shaft. In certain embodiments of the method, the articulating portion is configured to retain its shape when an external force is applied to the distal end. In particular embodiments, the external force is a radial force of approximately 0.5 pounds or less. In certain embodiments, the external force is an axial force of approximately 0.5 pounds or less. In particular embodiments of the method, the shaft is coupled to an insertion device comprising a positioning member configured to move the articulating portion of the shaft. In certain embodiments of the method, the insertion device comprises a locking member configured to lock the positioning member into a desired position. In specific embodiments of the method, inserting the therapeutic component into the paranasal sinus does not require the use of a guide wire or cannula. In particular embodiments, the paranasal sinus outflow tract comprises a maxillary, frontal or sphenoid sinus, and the therapeutic component is an inflatable balloon or a mechanical dilator. Specific embodiments comprise tracking the location of the therapeutic component with a location sensor.

Certain embodiments comprise: providing a stent disposed on the therapeutic component prior to inserting the therapeutic component into the paranasal sinus outflow tract; expanding the stent while expanding the therapeutic component; and withdrawing the therapeutic component from the stent so that the stent remains in the paranasal sinus outflow tract to maintain the dilated state for a period of time. In particular embodiments, the stent is bioabsorbable.

In certain embodiments, a bioabsorbable stent may be preferred to reduce the need for removal of the stent once the therapeutic effect has taken place, such as creating patency in the sinus opening throughout the healing period. In another embodiment, the stent may elude medications to create the therapeutic effect. These medications could include anti-inflammatory, antibiotic, steroid, etc. Since typical bioabsorbable stents are rigid, the stent could be composed of multiple leaflets that overlap in a slide and lock design to retain the shape of the ostium once inflated. Alternatively the stent could be composed of a magnesium based alloy that can retain its shape once expanded.

In exemplary embodiments, the stent device can be made of a biocompatible material. In particular embodiments, the stent device is made of a biodegradable material. In certain embodiments, the material is a biodegradable polymer. The material may be synthetic (e.g., polyesters, polyanhydrides) or natural (e.g., proteins, rubber, polysaccharides). In certain embodiments, the material is a homopolymer. In certain embodiments, the material is a co-polymer. In particular embodiments, the material is a block polymer. In other embodiments, the material is a branched polymer. In other embodiments, the material is a cross-linked polymer. In certain embodiments, the polymer is a polyester, polyurethane, polyvinyl chloride, polyalkylene (e.g., polyethylene), polyolefin, polyanhydride, polyamide, polycarbonate, polycarbamate, polyacrylate, polymethacrylate, polystyrene, polyurea, polyether, polyphosphazene, poly(ortho esters), polycarbonate, polyfumarate, polyarylate, polystyrene, or polyamine. In certain embodiments, the polymers is polylactide, polyglycolide, polycaprolactone, polydioxanone, polytrimethylene carbonate, and co-polymers thereof. Polymers that have been used in producing biodegradable implants and are useful in preparing the inventive devices include alpha-polyhydroxy acids; polyglycolide (PGA); copolymers of polyglycolide such as glycolide/L-lactide copolymers (PGA/PLLA), glycolide/D,L-lactide copolymers (PGA/PDLLA), and glycolide/trimethylene carbonate copolymers (PGA/TMC); polylactides (PLA); stereocopolymers of PLA such as poly-L-lactide (PLLA), poly-D,L-lactide (PDLLA), L-lactide/D,L-lactide copolymers; copolymers of PLA such as lactide/tetramethylglycolide copolymers, lactide/trimethylene carbonate copolymers, lactide/δ-valerolactone copolymers, lactide ε-caprolactone copolymers, polydepsipeptides, PLA/polyethylene oxide copolymers, unsymmetrically 3,6-substituted poly-1,4-dioxane-2,5-diones; polyhydroxyalkanate polymers including poly-beta-hydroxybutyrate (PHBA), PHBA/beta-hydroxyvalerate copolymers (PHBA/HVA), and poly-beta-hydroxypropionate (PHPA); poly-p-dioxanone (PDS); poly-δ-valerolatone; poly-r-caprolactone; methylmethacrylate-N-vinyl pyrrolidone copolymers; polyesteramides; polyesters of oxalic acid; polydihydropyrans; polyalkyl-2-cyanoacrylates; polyurethanes (PU); polyvinyl alcohol (PVA); polypeptides; poly-beta-maleic acid (PMLA); poly(trimethylene carbonate); poly(ethylene oxide) (PEO); poly(beta-hydroxyvalerate) (PHVA); poly(ortho esters); tyrosine-derived polycarbonates; and poly-beta-alkanoic acids. In certain embodiments, the polymer is a polyester such as poly(glycolide-co-lactide) (PLGA), poly(lactide), poly(glycolide), poly(D,L-lactide-co-glycolide), poly(L-lactide-co-glycolide), poly-β-hydroxybutyrate, and polyacrylic acid ester. In certain embodiments, the stent device is made of PLGA.

In certain embodiments, the stent device is made of 85% D,L-lactide and 15% glycolide co-polymer. In certain embodiments, the device is made of 50% D,L-lactide and 50% glycolide co-polymer. In certain embodiments, the device is made of 65% D,L-lactide and 35% glycolide co-polymer. In certain embodiments, the device is made of 75% D,L-lactide and 25% glycolide co-polymer. In certain embodiments, the device is made of 85% L-lactide and 15% glycolide co-polymer. In certain embodiments, the device is made of 50% L-lactide and 50% glycolide co-polymer. In certain embodiments, the device is made of 65% L-lactide and 35% glycolide co-polymer. In certain embodiments, the device is made of 75% L-lactide and 25% glycolide co-polymer. In certain embodiments, the stent device is made of poly(caprolactone). In certain embodiments, the device is made of Pebax, Polyimide, Braided Polyimide, Nylon, PVC, Hytrel, HDPE, or PEEK. In certain embodiments, the device is made of a fluoropolymer such as PTFE, PFA, FEP, and EPTFE. In certain embodiments, the device is made of latex. In other embodiments, the device is made of silicone. In certain embodiments, the polymer typically has a molecular weight sufficient to be shaped by molding or extrusion.

In certain embodiments, the stent device may also be composed of natural materials derived from human or animal sources. In specific embodiments, the allogeneic or human tissue grafts may be harvested from subjects other than the patient or from tissue banks For example, the xenogenic or animal tissue grafts can be derived from non-human species such as cows, pigs, etc.

In certain embodiments, allogeneic or xenogenic tissues, such as dermis, fascia, pericardium, cartilage, tendon, ligament and similar materials, may be useful for stent constructs. In specific embodiments, the intercellular matrixes of these tissues are processed to preserve the biological structure and composition, but the cells which may cause an immune response are removed. These constructs may then be processed into sheets or tubes to serve in a stenting function and are known to resorb by cell phagocytosis.

In particular embodiments, the stent may also comprise autologous or culture grown tissue. In specific embodiments, the tissues may be processed and terminally sterilized to enhance their biocompatibility and foreign response.

In certain embodiments, the device is made of a material that is bioabsorbed after the device is no longer needed. For example, the device may degrade after 1 week, 2 weeks, 3 weeks, 1 month, 2 months, 3 months, 4 months, 5 months, 6 months, 9 months, 1 year, 1.5 years, 2 years, 3 years, etc. The polymer used to make the device may be selected based on its degradation profile. The polymer can be selected as is known to the art to have a desired degradation period. For an implant of this invention, the degradation period may be up to about 2 years, or between about 6 months and about 1 year. As would be appreciated by one of skill in this art, the composition of the device may be varied to achieve the desired lifetime in vivo of the device. The device may be manufactured using a heat molding, injection molding, extrusion, cutting or laser cutting to obtain the necessary features.

Certain embodiments may include fenestrations or cut outs which need to be rigid and stiff enough to be inserted, expand if needed and then hold the tissues apart or ostium open. Furthermore, these features may also be strong and somewhat elastic so that they do not easily fracture during the process of implantation. To achieve that property, the device may be composed of a crystalline or amorphous polymer combined with an elastomeric polymer. For example, a highly crystalline polylactide may be blended with a polyhydroxybutarate; specifically 80-97% PLLA and 20-3% PHA. Similarly, caprolactone or trimethyl carbonate may be added to the crystalline polymer to make it more elastic. Elasticity of the construct can be achieved through the addition of the caprolactone or trimethyl carbonate to a lactide or glycolide monomer since the caprolactone and trimethyl carbonate have relatively low melting temperatures, i.e. −60° C. for carpolactone.

In certain embodiments, the stent may have a coating or incorporate a drug in the implant itself to provide the release of a pharmaceutical agent, which may prevent the adhesion of the stent in place, may prevent cell growth or scar formation, may enhance tissue healing, etc. In exemplary embodiments, the coating or incorporated drug may be biocompatible. In certain embodiments, the coating is a polymeric coating. In certain embodiments, the coating is a polymeric coating that includes a therapeutic agent. Classes of therapeutic agents that may be delivered by the stent include DNA, RNA, nucleic acids, proteins, peptides, or small molecules. Exemplary therapeutic agents include antibiotics, anti-inflammatory agents, corticosteroids, vasoconstrictors, vasodilators, anti-allergy agents, anti-histamines, cromolyn sodium, decongestants, asthma treatments, etc. In certain embodiments, the coating or incorporated drug may include retinoic acid to enhance mucosal wound healing. In certain embodiments, the coating includes cytotoxic agents such as paclitaxel to prevent cell growth on the stent. In other embodiments, the coating is Teflon. The stent may be coated with a polysaccharide such as hyaluronate.

Synthetic bioactive agents include but are not limited to growth factors such as platelet derived growth factor (PDGF), fibroblast growth factor (FGF), insulin-like growth factor (IGF), transforming growth factor beta (TGF-β), and other mitogenic or differentiation factors. Other synthetic bioactive agents could be small peptide analogues of the above-mentioned or other growth factors. Still other agents could be drugs or pharmacologically active substances which stimulate the growth or differentiation of tissue.

In certain embodiments, the stent may comprise anti-inflammatory and anti-infective agents, including for example, aminoglycosides, amphenicols, ansamycins, β-lactams, lincosamides, macrolides, nitrofurans, quinolones, sulfonamides, sulfones, tetracyclines, and any of their derivatives. In certain embodiments, β-lactams are the preferred antibacterial agents.

β-lactams that may be included in the stent implants include carbacephems, carbapenems, cephalosporins, cephamycins, monobactams, oxacephems, penicillins, and any of their derivatives. In certain embodiments, penicillins (and their corresponding salts) are the preferred β-lactams.

In particular embodiments, the penicillins that may be used in the biodegradable implants include amdinocillin, amdinocillin pivoxil, amoxicillin, ampicillin, apalcillin, aspoxicillin, azidocillin, azlocillin, bacampicillin, benzylpenicillinic acid, benzylpenicillin sodium, carbenicillin, carindacillin, clometocillin, cloxacillin, cyclacillin, dicloxacillin, epicillin, fenbenicillin, floxacillin, hetacillin, lenampicillin, metampicillin, methicillin sodium, mezlocillin, nafcillin sodium, oxacillin, penamecillin, penethamate hydriodide, penicillin G benethamine, penicillin G benzathine, penicillin G benzhydrylamine, penicillin G calcium, penicillin G hydrabamine, penicillin G potassium, penicillin G procaine, penicillin N, penicillin O, penicillin V, penicillin V benzathine, penicillin V hydrabamine, penimepicycline, phenethicillin potassium, piperacillin, pivampicillin, propicillin, quinacillin, sulbenicillin, sultamicillin, talampicillin, temocillin, and ticarcillin. In certain embodiments, amoxicillin may be included in the biodegradable implant. In particular embodiments, the biodegradable implant includes ampicllin. Penicillins combined with clavulanic acid such as Augmentin® (amoxicillin and clavulanic acid) may also be used.

Examples of antifungal agents that may be used in the biodegradable implants include allylamines, imidazoles, polyenes, thiocarbamates, triazoles, and any of their derivatives. In certain embodiments, imidazoles are the preferred antifungal agents.

In certain embodiments, if inclusion of an anti-inflammatory agent is desired, a steroidal anti-inflammatory agent, e.g., a corticosteroid, is employed. Examples of steroidal anti-inflammatory agents that may be used in the implants include 21-acetoxypregnenolone, alclometasone, algestone, amcinonide, beclomethasone, betamethasone, budesonide, chloroprednisone, clobetasol, clobetasone, clocortolone, cloprednol, corticosterone, cortisone, cortivazol, deflazacort, desonide, desoximetasone, dexamethasone, diflorasone, diflucortolone, difluprednate, enoxolone, fluazacort, flucloronide, flumethasone, flunisolide, fluocinolone acetonide, fluocinonide, fluocortin butyl, fluocortolone, fluorometholone, fluperolone acetate, fluprednidene acetate, fluprednisolone, flurandrenolide, fluticasone propionate, formocortal, halcinonide, halobetasol propionate, halometasone, halopredone acetate, hydrocortamate, hydrocortisone, loteprednol etabonate, mazipredone, medrysone, meprednisone, methylprednisolone, mometasone furoate, paramethasone, prednicarbate, prednisolone, prednisolone 25-diethylamino-acetate, prednisolone sodium phosphate, prednisone, prednival, prednylidene, rimexolone, tixocortol, triamcinolone, triamcinolone acetonide, triamcinolone benetonide, triamcinolone hexacetonide, and any of their derivatives. In certain embodiments, budesonide is included in the implant as the steroidal anti-inflammatory agent. In particular embodiments, the steroidal anti-inflammatory agent may be mometasone furoate. In some embodiments, the steroidal anti-inflammatory agent may be beclomethasone.

### BRIEF DESCRIPTION OF THE DRAWINGS

FIG. 1 illustrates a side view of a medical instrument including an insertion device, a flexible stylet, and a therapeutic assembly according to exemplary embodiments of the present disclosure.

FIG. 2 illustrates a side view of the insertion device of FIG. 1.

FIG. 3 illustrates a side view of the therapeutic assembly of FIG. 1.

FIG. 4 illustrates a side view of the flexible stylet of FIG. 1 in a first position.

FIG. 5 illustrates a side view of a flexible stylet with an adjustable luer connector.

FIG. 6 illustrates a side view of the flexible stylet of FIG. 5 in an insertion device.

FIG. 7 illustrates a side view of a flexible stylet and an insertion device with a universal clamp.

FIG. 8 illustrates a top view of the universal clamp of FIG. 7.

FIG. 9 illustrates a side view of a universal clamp of FIG. 7.

### DETAILED DESCRIPTION

Exemplary embodiments of the present disclosure provide systems, devices and methods for providing therapy to anatomical structures. In particular embodiments, the therapy comprises dilation of a paranasal sinus. Exemplary embodiments provide the ability to articulate an instrument and maintain the instrument in the articulated position when it is subjected to external forces. This rigidity of the articulated instrument can allow a user to extend the instrument into a paranasal ostium that may include granulation or scar tissue.

Multiple exemplary embodiments are disclosed in the description that follows. It is understood that various components of the disclosed embodiments can be combined to form additional exemplary embodiments. For example, a handle portion from one disclosed embodiment may be combined with a shaft of another disclosed embodiment. Such combinations are within the scope of this disclosure, which is not limited to the specific combinations of features and components illustrated in the exemplary embodiments.

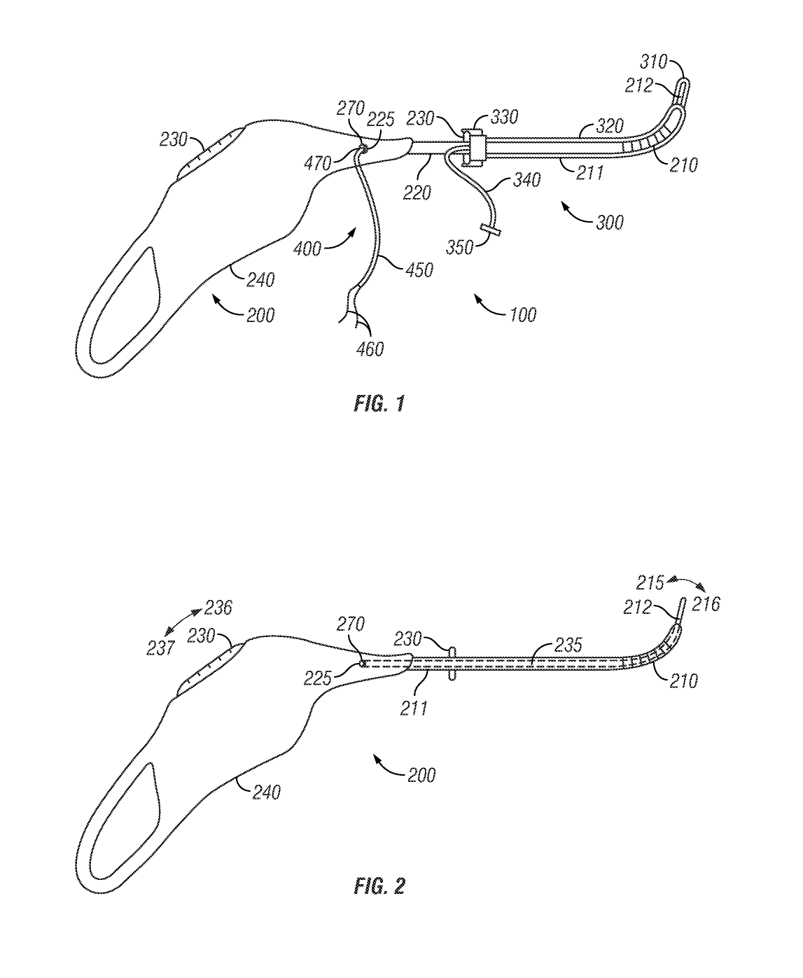

Referring initially to the exemplary embodiment shown in FIGS. 1-2, a medical instrument 100 comprises an insertion device 200, a therapeutic assembly 300, and a flexible stylet 400. In this embodiment, insertion device 200 comprises a handle portion 240 and a shaft 220. In the illustrated embodiments, shaft 220 comprises a first rigid portion 211 proximal to (and coupled to) handle portion 240. In the embodiments shown, shaft 220 also comprises an articulating section 210 and a second rigid portion 212 that are distal from handle portion 240, where articulating section 210 is located between first and second rigid portions 211 and 212. In exemplary embodiments, articulating section 210 may comprise articulating segments as disclosed in U.S. Pat. Nos. 7,553,275 and 7,670,284, incorporated herein by reference. It is understood that in other exemplary embodiments, shaft 220 may comprise a rigid shaft without an articulating section. In certain specific embodiments, shaft 220 may comprise a rigid shaft with an angled portion distal from the handle portion.

In the illustrated embodiments, insertion device 200 further comprises a positioning member 230 configured to articulate articulating section 210. Handle portion 240 of insertion device 200 also comprises an aperture 225 that provides access to a channel 235 extending within shaft 220. Insertion device 200 further comprises a coupling member 270 configured to couple flexible stylet 400 to insertion device 200. In exemplary embodiments, coupling member 270 may be configured to snap, compress, or otherwise positively engage flexible stylet 400 so that the relationship between flexible stylet 400 and insertion device 200 is fixed.

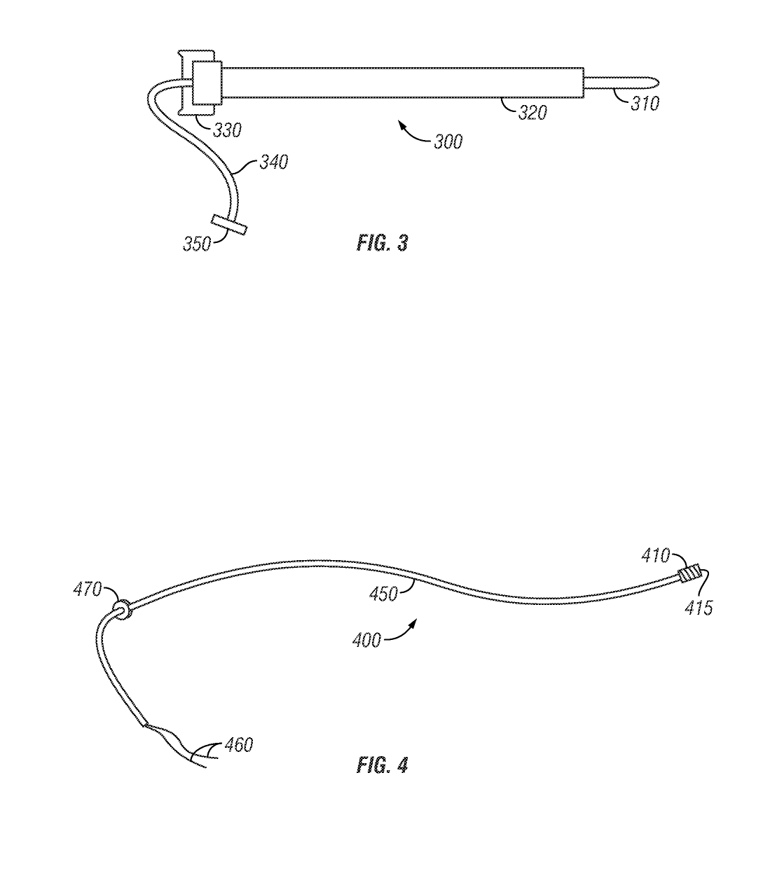

Referring now to FIG. 2, insertion device 200 is shown without therapeutic assembly 300 so that the articulation of articulating section 210 is visible. As shown in FIG. 3, a positioning member 230 can be rotated in the direction of arrows 236 and 237 in order to manipulate articulating section 210 in the direction of arrows 215 and 216. In certain embodiments, articulating section 210 may be configured to articulate in only one direction (e.g. in the direction of arrow 215 or arrow 216).

Referring now to FIG. 3, in this embodiment, therapeutic assembly 300 comprises a therapeutic component 310, a sleeve 320, and a conduit 340 coupled to a first coupling member 330 and a second coupling member 350. In certain embodiments therapeutic component 310 may comprise an expandable disposable medical device. In exemplary embodiments, second coupling member 350 may be coupled to a pressurizing member (not shown) including, for example, a syringe. In certain embodiments, therapeutic assembly 300 may be configured to expand therapeutic component 310, and/or deliver fluids to therapeutic component.

Referring now to FIG. 4, flexible stylet 400 is shown removed from insertion device 200. As shown in this embodiment, flexible stylet 400 comprises a locating device 410 near a distal end 415. In specific embodiments, locating device 410 may comprise a transmitter configured to transmit a signal to an image guidance system. In other embodiments, locating device 410 may comprise an active or passive sensor, including for example a ferrous metal coil, configured to sense or receive a signal to an image guidance system. In particular embodiments locating device 410 may be configured as an electromagnetic, ultrasonic, or electrostatic transmitter or sensor.

Prior to (or during) use of medical instrument 100, flexible stylet 400 may be inserted into aperture 225 of insertion device 200 (e.g., by initially inserting distal end 415 and location device 410 into aperture 225). Flexible stylet 400 may be further inserted such that distal end 415 and location device 410 are inserted into and along channel 235 until the flexible stylet coupling member 470 engages coupling member 270. With coupling members 270 and 470 engaged and coupled, the relationship between flexible stylet 400 and insertion device 200 can be fixed in a known position.

Flexible stylet 400 also comprises a flexible portion 450 extending between coupling member 470 and locating device 410. In the embodiment shown, flexible stylet 400 also comprises electrical coupling members 460 (e.g. electrical leads with suitable coupling mechanisms) to transmit electrical signals through flexible stylet 400. In exemplary embodiments, electrical coupling members 460 can be coupled to an image guidance system to track the position of locating device 410 while it is located within an anatomical opening (e.g. a paranasal sinus).

Referring back now to FIG. 1, therapeutic assembly 300 has been coupled to insertion device 200 and flexible stylet has been inserted into channel 235. In the position shown in FIG. 2, shaft 220 and articulating section 210 have been inserted into first coupling member 330, sleeve 320 and therapeutic component 310 such that therapeutic component 310 is disposed proximal to articulating section 210 and second rigid portion 212. In this embodiment sleeve therapeutic component 310 are transparent or translucent so that portions of shaft 220 are visible with sleeve 320 and therapeutic component 310. It is understood that in other embodiments, sleeve 320 and/or therapeutic component 310 may not be transparent or translucent. Flexible stylet has also been inserted into aperture 225 and channel 235 so that coupling members 270 and 470 are coupled.

During use, insertion device 200 can be positioned so that shaft 220 is inserted through an ostium and into a paranasal sinus or other anatomical opening or body lumen. In specific embodiments, second rigid portion 212 and a portion of articulating section 210 can initially be inserted into a nostril of a patient. Shaft 220 can be further inserted so that a portion of first rigid portion 211 (e.g. the portion proximal to articulating section 210) is also inserted into the nostril. In specific embodiments, positioning member 230 can be manipulated as shaft 220 is inserted so that articulating section 210 is articulated to a desired position and second rigid portion 212 is oriented as desired by the user. Such a configuration can allow the user to insert second rigid portion 212 (and therapeutic component 310) into the desired location.

As previously noted, flexible stylet 400 can be inserted and removed from channel 235 of shaft 200. This can allow a user to track the location of locating device 410 via an image guidance system as desired by inserting flexible stylet 400 (and locating device 410) from channel 235 of insertion device 200 when the user desired to use the image guidance system. Locating device 410 is not permanently mounted or affixed to insertion device 200, and therefore can be separated from medical instrument 100 and insertion device 200 when the user does not wish to use the image guidance system. For example, when locating device 410 is placed outside of the portion of the operating field covered by the electromagnetic field, the image guidance system tracking function can be temporarily disabled and the display removed from the monitor screen. This can allow the user to view other information or displays on the screen as desired.

The ability to remove flexible stylet 400 from insertion device 200 can also allow other instruments to be inserted into channel 235 to perform other functions during use of insertion device 200. For example, therapeutic instruments that can provide suction, irrigation, drug delivery, or laser, radio or ultrasonic frequency therapy can be inserted into channel 235 as desired. Other examples of therapeutic instruments that may be inserted into channel 235 may perform therapeutic agent (e.g., drug) delivery, tissue cutting, tissue grasping, or balloon dilation. The ability to insert into channel 235 and remove the various instruments from channel 235 allows multiple instruments to utilize a single channel, thereby minimizing the cross-section of shaft 220 by eliminating the need for multiple channels. This can allow shaft 220 to be inserted into smaller and less accessible anatomical openings.

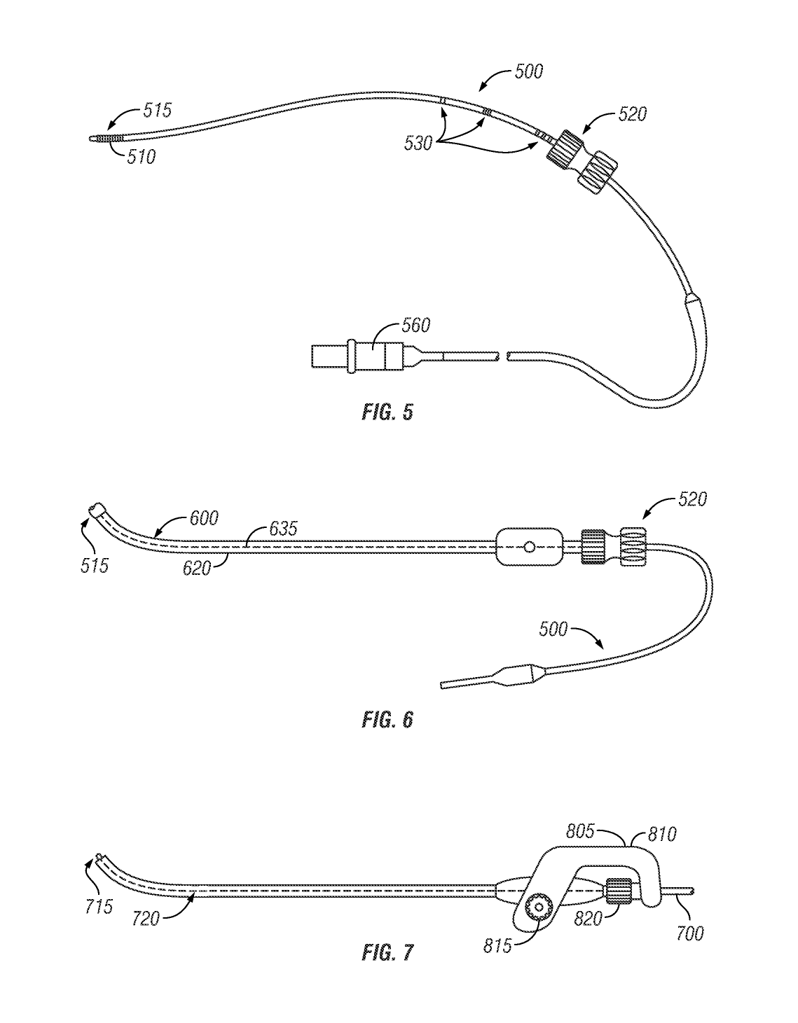

Referring now to FIG. 5, a flexible stylet 500 according to another embodiment is shown removed from an insertion device. As shown in this embodiment, the flexible stylet 500 comprises a locating device 510 near a distal end 515. The locating device 510 may be configured as previously described with respect to locating device 410. An adjustable luer connector 520 is disposed along the length of the flexible stylet 500. The adjustable luer connector 520 can be fixedly clamped at a specific location along the length of the flexible stylet 500. The adjustable luer connector 520 can also be used to connect the flexible stylet to another device using screw attachment threads or the like. One suitable adjustable luer connector 520 is a Touhy Borst connector. The flexible stylet 500 also comprises electrical coupling member 560 (e.g. electrical leads with suitable coupling mechanisms) to transmit electrical signals through flexible stylet 500 and to couple the flexible stylet 500 to an image guidance system.

Referring now to FIG. 6, prior to (or during) use of an insertion device 600, flexible stylet 500 may be inserted into an aperture (not shown) of insertion device 600 (e.g., by initially inserting distal end 515 and location device 510 into the aperture). Flexible stylet 500 may be further inserted such that distal end 515 and location device 510 are inserted into and along channel 635 of shaft 620 until the location device 510 is located at a desired location, such as the distal end of the shaft 620. Preferably, the shaft 620 is a malleable or articulatable shaft. Once the location device 510 is disposed at a desired location with respect to the flexible stylet 500, the adjustable luer connector 520 is clamped to the flexible stylet 500 to maintain the relationship between flexible stylet 400 and insertion device 600 in a known position. Indicia 530 such as marker bands may be placed on the flexible stylet 600 to provide a visual indicator of where the luer connector should be placed for various insertion devices.

The method of using the flexible stylet 500 is substantially similar to the method described above with respect to flexible stylet 400. However, the use of an adjustable luer connector 520 allows the flexible stylet 500 to be used with a variety of insertion devices 600 of varying lengths.

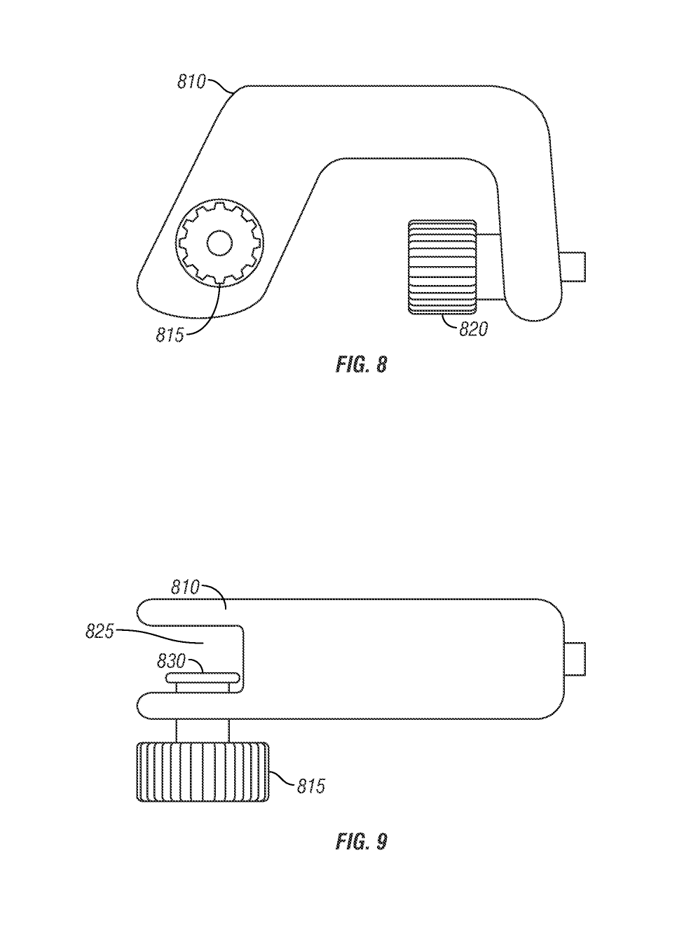

Referring now to FIGS. 7-9, a flexible stylet 700 according to another embodiment is shown inserted into an insertion device 720. As shown in this embodiment, the flexible stylet 700 comprises a locating device (not shown) near a distal end 715 of the stylet. The locating device may be configured as previously described with respect to locating device 410. A universal clamp 805 is disposed on the insertion device 720. The universal clamp 805 comprises a clamp arm 810 which has an opening 825 for receiving an insertion device. The opening 825 is placed at a suitable position on an insertion device, such as shown in FIG. 7, and is fixed into place by rotating the rotatable knob 815 so that the foot 830 of the rotatable knob is pressed against the insertion device to fix it into place.

The clamp arm 810 includes a fitting 820 with an aperture. The flexible stylet 700 may be placed through the aperture and adjusted so that the locating device is disposed at a desired location. Once the locating device is at the desired location, the fitting 820 is tightened to fix the flexible stylet 700 into place. In one embodiment, the fitting is rotated to tighten it. The universal clamp 800 allows the use of the adjustable length flexible stylet 700 with a variety of different medical devices.

Certain embodiments also comprise specific methods of using the therapeutic components described herein. For example, certain methods may comprise preparing a target sinus, including if needed, performing surgical debridement as required to obtain adequate access and visualization. The methods may also comprise coupling a therapeutic component to a pressuring device and to a first insertion device. The methods may further comprise inserting the therapeutic component into a first nasal passageway and a first sinus, using articulation of the first delivery device and visualization via an endoscope to locate the therapeutic component if needed. In certain embodiments, the therapeutic component is positioned with the aid of an image guidance navigation system via a location sensor coupled to the insertion device. In such embodiments, the articulating insertion device can be configured to provide rigidity at pre-set positions to provide the accuracy needed for navigation technology. In certain embodiments, the extended flexible therapeutic component is positioned with the aid an image guidance navigation system via a micro-location sensor coupled to the distal tip of the therapeutic component. In certain embodiments, the therapeutic component may be placed in the desired location without the use of a cannula or guide wire.

Additionally, exemplary methods may comprise expanding and contracting the therapeutic component to dilate the target sinus, for example by inflating a dilation balloon. The method may further comprise observing the first sinus with the endoscope, and expanding and contracting the therapeutic component again as needed in order to obtain the desired expansion of the first sinus, and/or to insert the therapeutic component into a second sinus and expanding and contracting the therapeutic component to obtain the desired expansion of a second sinus. Certain embodiments may also comprise removing the therapeutic component from the delivery device and coupling the therapeutic component to a second delivery device; and repeating the previously-described actions with a second sinus.

Specific embodiments may also comprise placing a therapeutic component into a target sinus structure using an insertion device and then removing the insertion device from the sinus while leaving the therapeutic component in the sinus. The therapeutic component may then be expanded (e.g, inflated) using a pressurizing member. The therapeutic component may then be returned to its non-expanded state (e.g. by venting the pressurizing member) and retrieved from the sinus using a tether or a conduit between the pressurizing member and the therapeutic component. One potential advantage of such an embodiment is that a single operator may perform the expansion/dilation procedure. A first operator does not have to hold the insertion device while a second operator expands the therapeutic component.

In certain embodiments, a method of use comprises coupling a therapeutic component to a flexible endoscope. This arrangement can allow the endoscope image to be used for visualization and placement of the therapeutic component without surgical debridement. In addition a light on the endoscope may be utilized to transilluminate the sinus (allowing the user to see the light externally) to assist in correct placement of the therapeutic component. In certain embodiments, a therapeutic component may be placed without external visualization or transillumination. In other methods, the therapeutic component and endoscope may be coupled to an articulating instrument to assist in delivery and positioning of the therapeutic component using visualization from the endoscope.

Certain methods of use may also include the placement of an expandable stent in a sinus structure. For example, a user may initially debride or dilate a target sinus as needed and then insert a stent and therapeutic component into a sinus. The therapeutic component may be expanded (e.g. via a pressurizing member) to expand and deploy the stent in the desired location within the sinus. In certain embodiments, an endoscope may be used to verify adequate deployment of the stent. If needed, the stent may be further expanded with a larger therapeutic component. In certain embodiments, the stent may be self-expanding and may be expanded when a retention sleeve is removed after placement within the sinus.

In alternate embodiments, the method of use may additionally include delivery of a therapeutic agent such as an antibiotic spray, powder or solution into the paranasal sinus. This agent delivery may be done before, during, or after performing a therapy on the sinus passageway. For example, a user may deliver a solution through a secondary lumen of the therapeutic component into the frontal sinus during balloon dilation of the frontal sinus recess. In this manner, the balloon both dilates the passage and blocks drainage of the solution, such that the solution remains in the frontal sinus for a period of time while the balloon is inflated.

### Equivalents and Scope

The foregoing has been a description of certain non-limiting preferred embodiments of the invention. Those skilled in the art will recognize, or be able to ascertain using no more than routine experimentation, many equivalents to the specific embodiments of the invention described herein. Those of ordinary skill in the art will appreciate that various changes and modifications to this description may be made without departing from the spirit or scope of the present invention, as defined in the following claims.

In the claims articles such as “a”, “an”, and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention also includes embodiments in which more than one, or all of the group members are present in, employed in, or otherwise relevant to a given product or process. Furthermore, it is to be understood that embodiments of the invention encompasses all variations, combinations, and permutations in which one or more limitations, elements, clauses, descriptive terms, etc., from one or more of the claims or from relevant portions of the description is introduced into another claim. For example, any claim that is dependent on another claim can be modified to include one or more limitations found in any other claim that is dependent on the same base claim. Furthermore, where the claims recite a composition, it is to be understood that methods of using the composition for any of the purposes disclosed herein are included, and methods of making the composition according to any of the methods of making disclosed herein or other methods known in the art are included, unless otherwise indicated or unless it would be evident to one of ordinary skill in the art that a contradiction or inconsistency would arise. In addition, embodiments of the invention encompasses compositions made according to any of the methods for preparing compositions disclosed herein.

Where elements are presented as lists, e.g., in Markush group format, it is to be understood that each subgroup of the elements is also disclosed, and any element(s) can be removed from the group. It is also noted that the term “comprising” is intended to be open and permits the inclusion of additional elements or steps. It should be understood that, in general, where the invention, or aspects of the invention, is/are referred to as comprising particular elements, features, steps, etc., certain embodiments of the invention or aspects of the invention consist, or consist essentially of, such elements, features, steps, etc. For purposes of simplicity those embodiments have not been specifically set forth in haec verba herein. Thus for each embodiment of the invention that comprises one or more elements, features, steps, etc., the invention also provides embodiments that consist or consist essentially of those elements, features, steps, etc.

Where ranges are given, endpoints are included. Furthermore, it is to be understood that unless otherwise indicated or otherwise evident from the context and/or the understanding of one of ordinary skill in the art, values that are expressed as ranges can assume any specific value within the stated ranges in different embodiments of the invention, to the tenth of the unit of the lower limit of the range, unless the context clearly dictates otherwise. It is also to be understood that unless otherwise indicated or otherwise evident from the context and/or the understanding of one of ordinary skill in the art, values expressed as ranges can assume any subrange within the given range, wherein the endpoints of the subrange are expressed to the same degree of accuracy as the tenth of the unit of the lower limit of the range.

In addition, it is to be understood that any particular embodiment of the present invention may be explicitly excluded from any one or more of the claims. Any embodiment, element, feature, application, or aspect of the compositions and/or methods of the invention can be excluded from any one or more claims. For purposes of brevity, all of the embodiments in which one or more elements, features, purposes, or aspects is excluded are not set forth explicitly herein.

### REFERENCES

The entire disclosures of the following references are incorporated by reference herein:
U.S. Pat. No. 2,525,183
U.S. Pat. No. 4,733,665
U.S. Pat. No. 4,740,207
U.S. Pat. No. 4,877,030;
U.S. Pat. No. 4,954,126
U.S. Pat. No. 5,007,926;
U.S. Pat. No. 5,059,211
U.S. Pat. No. 5,192,307
U.S. Pat. No. 5,421,955
U.S. Pat. No. 5,441,515
U.S. Pat. No. 5,443,500
U.S. Pat. No. 5,549,662
U.S. Pat. No. 5,618,299
U.S. Pat. No. 5,643,314
U.S. Pat. No. 5,649,977
U.S. Pat. No. 5,733,328
U.S. Pat. No. 5,735,872
U.S. Pat. No. 7,462,175
U.S. Pat. No. 7,500,971
U.S. Pat. No. 7,553,275
U.S. Pat. No. 7,670,284
U.S. Pat. Pub. No. 2004/0064150
U.S. Pat. Pub. No. 2009/0125046
U.S. Pat. Pub. No. 2008/0215083
U.S. Pat. Pub. No. 2008/0208242
U.S. Pat. Pub. No. 2008/0215082
U.S. Pat. Pub. No. 2008/0208243
U.S. Pat. Pub. No. 2006/0149310
U.S. Pat. Pub. No. 2006/0136041
U.S. Pat. Pub. No. 2010/0312338
Göttmann, D., Strohm, M., Strecker, E. P., Karlsruhe, D. E., “Balloon dilatation of Recurrent Ostial Oclusion of the Frontal Sinus”, Abstract No. B-0453, European Congress of Radiology (2001)
Strohm, M., Göttmann, D., “Treatment of Stenoses of Upper Air Routes by Balloon Dilation”, Proceeding of the 83^{rd} Annual Convention of the Association of West German ENT Physicians (1999).
Balcon et al., “Recommendations on Stent Manufacture, Implantation and Utilization,” European Heart Journal (1997), vol. 18, pages 1536-1547.
“The Stenter's Notebook,” Physician's Press (1998), Birmingham, Mich.

What is claimed is:

1. A medical instrument configured for insertion into a paranasal sinus, the medical instrument comprising:
an insertion device comprising:
a handle portion;
a shaft comprising a channel located within the shaft; and
a flexible stylet comprising a locating device, wherein the flexible stylet is configured for insertion into the channel and removal from the channel.

2. The medical instrument of claim 1, wherein the shaft comprises an articulating section and a first rigid portion between the handle portion and the articulating section.

3. The medical instrument of claim 2, wherein the shaft further comprises a second rigid portion and wherein the articulating section is between the first and second rigid portions.

4. The medical instrument of claim 2, wherein the insertion device comprises a positioning member configured to move the articulating section of the shaft.

5. The medical instrument of claim 1, wherein the locating device comprises a transmitter.

6. The medical instrument of claim 5, wherein the transmitter is an electromagnetic, ultrasonic, electrostatic transmitter.

7. The medical instrument of claim 1, wherein the locating device comprises a sensor.

8. The medical instrument of claim 7, wherein the sensor is an electromagnetic, ultrasonic, electrostatic sensor.

9. The medical instrument of claim 1, wherein the flexible stylet comprises a coupling mechanism configured to couple the flexible stylet to the insertion device.

10. The medical instrument of claim 9, wherein the coupling mechanism comprises an adjustable luer lock connector.

11. The medical instrument of claim 10, wherein the adjustable luer lock connector is a Touhy Borst connector.

12. The medical instrument of claim 1, further comprising a clamp for fixing the location of the flexible stylet with respect to the insertion device.

13. The medical instrument of claim 1, wherein the insertion device comprises a coupling mechanism configured to couple the flexible stylet to the insertion device.

14. The medical instrument of claim 1, wherein the handle portion of the insertion device comprises an aperture configured to receive the flexible stylet.

15. The medical instrument of claim 1, further comprising a therapeutic assembly comprising a sleeve and a therapeutic component configured such that the shaft can be inserted into the sleeve and the therapeutic component.

16. The medical instrument of claim 1, further comprising a therapeutic instrument configured for insertion into the channel.

17. The medical instrument of claim 16, wherein the therapeutic instrument is configured to provide therapy comprising laser, radio or ultrasonic frequency therapy, tissue cutting, tissue grasping, or balloon dilation.

18. The medical instrument of claim 1, further comprising a therapeutic instrument configured to provide therapy through the channel.

19. The medical instrument of claim 18, wherein the therapeutic instrument provides suction, irrigation, or drug delivery.

20. The medical instrument of claim 1, further comprising a therapeutic assembly having a sleeve and a therapeutic component, wherein the shaft is configured for insertion into the sleeve and the therapeutic component.

21. The medical instrument of claim 21, wherein the therapeutic component is a balloon.

22. A method of treating a paranasal sinus, the method comprising:
obtaining an insertion device comprising a handle portion and a shaft portion;
positioning a distal end of the shaft portion of the insertion device proximal to the paranasal sinus, wherein the shaft portion comprises a channel extending through the shaft;
inserting a flexible stylet comprising a locating device into the channel; and
removing the flexible stylet from the channel.

23. The method of claim 22, further comprising inserting a therapeutic instrument into the channel.

24. The method of claim 23, further comprising using the therapeutic instrument to provide suction, irrigation, drug delivery, laser, radio or ultrasonic frequency therapy drug delivery, tissue cutting, tissue grasping, or balloon dilation.

25. The method of claim 22, wherein the locating device is configured to communicate with an image guidance system.

26. The method of claim 22, wherein the locating device transmits a signal to an image guidance system.

27. The method of claim 22, wherein the locating device senses a signal transmitted from an image guidance system.

28. The method of claim 22, further comprising:
coupling the flexible stylet to the insertion device via a coupling mechanism after inserting the flexible stylet; and
decoupling the flexible stylet from the insertion device prior to removing the flexible stylet from the channel.

29. The method of claim 22, further comprising adjusting the position of the location device prior to coupling the flexible stylet to the insertion device.

30. The method of claim 24, further comprising using the locating device to locate the distal end of the shaft portion via an image guidance system when the distal end of the shaft portion is inserted into the paranasal sinus.

31. The method of claim 22, wherein the shaft portion comprises an articulating portion and a positioning member configured to articulate the articulating portion.

32. The method of claim 31, further comprising manipulating the positioning member to articulate the articulating portion when a distal end of the shaft portion is proximal to a paranasal sinus.
